(19) United States
(12) Patent Application Publication
(10) Pub. No.: US 2025/0342963 A1
(43) Pub. Date: Nov. 6, 2025

(54) BOOSTING THE PREDICTIVE POWER OF VIRUS-SPECIFIC T-CELL (VST) CLINICAL TRIALS VIA GENERATIVE MODELS

(71) Applicant: Children's National Medical Center, Washington, DC (US)

(72) Inventors: Pamela Chansky, Washington, DC (US); Wei Li, Rockville, MD (US); Michael Keller, Laurel, MD (US)

(73) Assignee: Children's National Medical Center, Washington, DC (US)

(21) Appl. No.: 19/199,170

(22) Filed: May 5, 2025

Related U.S. Application Data
(60) Provisional application No. 63/643,246, filed on May 6, 2024.

(51) Int. Cl. G16H 50/20; G16B 5/00

(52) U.S. Cl. CPC G16H 50/20; G16B 5/00

(57) ABSTRACT

A method for treating an immunocompromised patient including collecting from patient values for at least one variable selected from the group, inputting the values for the one or more variables to a neural network (NN) performed on one or more computers to produce a score indicative of likelihood of response or non-response to an anti-viral drug and a score indicative of likelihood of response or non-response to Virus-Specific T-Cells (VSTs) and administering an antiviral drug to a patient who has a threshold score indicative of a likelihood of a response to anti-viral therapy and administering VSTs to a patient who has a threshold score indicative of a likelihood of a response to VST therapy.

### CROSS-REFERENCE TO RELATED APPLICATIONS

This present disclosure claims the benefit of U.S. Provisional Application No. 63/643,246, filed on May 6, 2024, which is incorporated herein by reference in its entirety.

### FIELD OF THE INVENTION

The present disclosure relates to predicting efficacy of treatments including virus-specific T-cell (VST) treatments by applying generative models.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Choosing the right treatments for individual patients is critical to maximize the treatment effect. The decision of selecting patients for certain treatments, such as VST treatments versus continuing with additional course or courses of antiviral therapy is particularly challenging. The lack of large datasets, complex underlying relationships between clinical variables, and variations in how response may be determined, all contribute to the difficult nature of this problem.

### BRIEF DESCRIPTION

Note that this summary section does not specify every embodiment and/or incrementally novel aspect of the present disclosure or claimed invention. Instead, this summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

1. A method for treating an immunocompromised patient comprising: (a) collecting from patient values for one or more variables, comprising selecting prior cancer remission or relapse, prior reaction after transplant, a degree of HLA match, type of viral infection, type of comorbidity or infection, viral load, prior receipt of one or more immunosuppressive medications, prior receipt of one or more anti-cancer medications, or prior receipt of one or more antiviral medications; (b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and (c) administering an antiviral drug to a patient who has a threshold score indicative of a likelihood of a therapeutic response to anti-viral therapy; and/or administering VSTs to a patient who has a threshold score indicative of a likelihood of a therapeutic response to VST therapy.

2. The method for treating an immunocompromised patient according to embodiment 1 further comprising: (a) collecting from the patient values for one or more variables selected from the group comprising: transplant donor and recipient age and sex, presence or absence of inborn error of immunity, malignant, non-malignant hematology condition, or other primary immunodeficiency, upon original diagnosis, presence or absence of partial or complete cancer remission including no detectable cancer, reduction or growth of a tumor, higher or lower number of cancer cells compared to prior levels, and symptomatic improvement or regression compared to a prior level, prior graft-vs-host reaction after transplant, presence or absence of myeloablative conditioning regiment (MA), reduced intensity conditioning regimen RIC), or no conditioning regiment (NMA), transplant donor type including mismatched related donor, matched related donor, matched unrelated donor, umbilical cord cell transplant, or no donor, a degree of HLA match ranging from 1 to 6 based on the number of major alleles shared, wherein said major alleles include HLA-A, HLA-B, HLA-C and HLA-DR, HLA-DQ and HLA-DP, cellular depletion or ablation of TCRαβ, CD19, naive T cells (CD45RA+ T cells) and/or CD34+ T cells, a level of CD8+ or CD8+ T cells or a ratio of CD4+ cells to CD8+ T cells or a higher or lower level compared to a prior level, type of viral infection comprising adenovirus (AdV), Epstien-Barr Virus (EBV), Cytomegalovirus (CMV), Herpes Simplex Virus (HSV), human herpes virus 8, Varicella-Zoster virus, or human papillomarvirus, type of comorbidity or infection caused by an opportunistic virus, bacterium, fungi, or parasite, viral load at a time of infusion of antiviral drug or VST, wherein viral load can be measured in IU/ml by PCR, prior receipt of one or more immunosuppressive medications comprising systemic corticosteroids, Budesonide, Tacrolimus (FK), Cyclosporine (CsA), Mycophenolic acid (MMF), Sirolimus, Anti-thymocyte globulin (ATG), Alemtuzumab (Campath), antivirals including Ganciclovir, Valganciclovir, Foscarnet, Cidofovir, Brincidofovir, or Rituximab, prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor, prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir; (b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and (c) administering an antiviral drug to a patient who has a threshold score indicative of a likelihood of a therapeutic response to anti-viral therapy; and/or administering VSTs to a patient who has a threshold score indicative of a likelihood of a therapeutic response to VST therapy.

3. The method of embodiment 1 or 2, wherein the immunocompromised patient is infected by an opportunistic virus and is administered an antiviral drug and/or VST.

4. The method of embodiment 1, 2 or 3, wherein the immunocompromised patient is infected by cytomegalovirus, Epstein-Barr virus, or Adenovirus; and wherein the patient is administered Ganciclovir, Valganciclovir, Foscarnet, Cidofovir, Brincidofovir, Acyclovir and Rituximab and/or administered VSTs that recognize Cytomegalovirus, Epstein-Barr virus, and/or Adenovirus.

5. The method of any one of embodiments 1-4, wherein the immunocompromised patient has undergone a autograft, an allograft, or a xenograft and is infected by an opportunistic virus and is administered an antiviral drug and/or VST.

6. The method of any one of embodiments 1-5, wherein the immunocompromised patient has undergone a autograft, an allograft, or a xenograft and is infected by cytomegalovirus, Epstein-Barr virus, or Adenovirus; wherein the patient is administered Ganciclovir, Valganciclovir, Foscarnet, Cidofovir, Brincidofovir, Acyclovir and Rituximab and/or administered VSTs that recognize cytomegalovirus, Epstein-Barr virus, and/or Adenovirus.

7. The method of embodiment 6, wherein the autograft, allograft or xenograft is bone marrow cells or stem cells.

8. The method of any one of embodiments 1-7, wherein the immunocompromised patient has undergone a autograft, an allograft, or a xenograft, has been administered an immunosuppressant, and is infected by cytomegalovirus, Epstein-Barr virus, or Adenovirus; wherein the patient is administered Ganciclovir, Valganciclovir, Foscarnet, Cidofovir, Brincidofovir, Acyclovir and Rituximab and/or administered VSTs that recognize cytomegalovirus, Epstein-Barr virus, and/or Adenovirus.

9. The method of embodiment 8, wherein the immunosuppressant comprises Budesonide GI, Tacrolimus (FK), Mycophenolate mofetil (MMF), Sirolimus, Infliximad, Vedolizumad, Anti-thymocyte globulin (ATG) and Alemtuzumab (Campath).

10. The method of any one of embodiments 1-9, wherein the patient has a primary or secondary immunodeficiency, is infected by an opportunistic virus, and is administered an antiviral drug and/or VST.

11. The method of any one of embodiments 1-10, wherein the patient has a secondary immunodeficiency that comprises infection by HIV, a burn, drug abuse, chemotherapy, radiation therapy, diabetes millitus, malnutrition, or leukemia or other cancer of the immune system, viral hepatitis or other immune complex disease, or multiple myeloma.

12. The method of any one of embodiments 1-11, wherein the NN includes a first NN model and a second NN model cascaded to the first NN model, and inputting the values for the one or more variables to the NN to produce the score includes: inputting the values for the one or more variables to the first NN model to generate synthetic data that are in a larger amount than the values of the one or more variables; and inputting the synthetic data to the second NN model to produce the score.

13. The method of embodiment 12, wherein the first NN model comprises a generative artificial intelligence (genAI) model, wherein the genAI model comprises a variational autoencoder (VAE) model, a generative adversarial network (GAN) model, or a Gaussian copula synthesizer (GC) model.

14. The method of embodiment 12, wherein the second NN model comprises a logistic regression (LR) model, a naïve Bayes (NB) model, and/or a support vector machine (SVM) model.

15. The method of embodiment 12, further comprising: determining similarity of the synthetic data to the original data, wherein the second NN model is trained with the synthetic data if the similarity of the synthetic data is over a similarity threshold in terms of the distribution to the original data.

16. The method of embodiment 15, wherein the similarity of the synthetic data is accessed by Total Variation Distance complement, Kolmogorov-Smirnov complement, or Spearman correlations.

17. The method of any one of embodiments 1-16, further comprising: identifying at least one of the variables that contributes most to a predictive ability of the therapeutic approach.

18. The method of embodiment 12, further comprising: inputting training values for the one or more variables to the first NN model to generate training synthetic data that are in a larger amount than the training values of the one or more variables; and training the second NN with the training synthetic data.

19. The method of any one of embodiments 1-18, wherein the one or more variables include continuous, binary and/or categorical variables.

20. The method of embodiment 19, wherein the values of the categorical variables are one-hot encoded prior to modeling.

21. The method of embodiment 19, wherein the values of the continuous variables are log normalized.

22. A method for treating an immunocompromised patient in need of an anti-cancer medication and/or in need of an anti-viral medication comprising: (a) collecting from patient values for one or more variables, comprising selecting prior cancer remission or relapse, prior reaction after transplant, a degree of HLA match, type of viral infection, type of comorbidity or infection, viral load, prior receipt of one or more immunosuppressive medications, prior receipt of one or more anti-cancer medications, or prior receipt of one or more antiviral medications, (b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and (c1) administering an antiviral drug to a patient who has a threshold score indicative of a likelihood of a therapeutic response to anti-viral therapy, and/or (c2) administering an anti-cancer drug to a patient who has a threshold score indicative of a likelihood of a therapeutic response to anti-cancer therapy; and/or (c3) administering VSTs to a patient who has a threshold score indicative of a likelihood of a therapeutic response to VST therapy.

23. The method of embodiment 22, further comprising: (a) collecting from the patient values for at least one variable selected from the group comprising: prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor, or prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir; (b) inputting the values for the one or more variables to a neural network (NN) performed on one or more computers to produce (i) a score indicative of likelihood of response, non-response, or anti-therapeutic response to an anti-viral drug and/or (ii) a score indicative of likelihood of response, non-response, or antitherapeutic response to Virus-Specific T-Cells (VSTs); and (c) administering an antiviral drug to a patient who has a threshold score indicative of a likelihood of a therapeutic response to anti-viral therapy; and/or administering VSTs to a patient who has a threshold score indicative of a likelihood of a therapeutic response to VST therapy.

24. The method according to embodiment 22 or 23, wherein the group further comprises: presence or absence of inborn error of immunity, malignant, non-malignant hematology condition, or other diagnosis, upon original diagnosis, presence or absence of partial or complete cancer remission including no detectable cancer, reduction or growth of a tumor, higher or lower number of cancer cells compared to prior levels, and symptomatic improvement or regression compared to a prior level, prior cancer relapse, transplant donor and recipient age and sex, prior graft-vs-host reaction after transplant, or myeloablative conditioning regiment (MA), reduced intensity conditioning regimen RIC), or no conditioning regiment (NMA), transplant donor type including mismatched related donor, matched related donor, matched unrelated donor, umbilical cord cell transplant, or no donor.

25. The method according to embodiment 22, 23 or 24, wherein the group further comprises: a degree of HLA match ranging from 1 to 6 based on the number of major alleles shared, wherein said major alleles include HLA-A, HLA-B, HLA-C and HLA-DR, HLA-DQ and HLA-DP, cellular depletion or ablation of TCRαβ, CD19, naive T cells (CD45RA+ T cells) and/or CD34+ T cells, a level of CD8+ or CD8+ T cells or a ratio of CD4+ cells to CD8+ T cells or a higher or lower level compared to a prior level, a type of viral infection comprising adenovirus (AdV), Epstien-Barr Virus (EBVCytomegalovirus (CMV), Herpes Simplex Virus (HSV), human herpes virus 8, Varicella-Zoster virus, or human papillomarvirus, or a type of comorbidity or infection caused by an opportunistic bacterium, fungi, or parasite, a viral load at a time of infusion measured in IU/ml by PCR.

26. The method according to any one of embodiments 22-25, wherein the variable group further comprises: prior receipt of one or more immunosuppressive medication comprising systemic corticosteroids, Budesonide, Tacrolimus (FK), Cyclosporine (CsA), Mycophenolic acid (MMF), Sirolimus, Anti-thymocyte globulin (ATG), Alemtuzumab (Campath), or prior receipt of one or more antivirals including Ganciclovir, Valganciclovir, Foscarnet, Cidofovir, Brincidofovir, and Rituximab.

27. The method according to any one of embodiments 22-26, wherein the variable group further comprises: prior receipt of one or more immunosuppressive medication comprising systemic corticosteroids, Budesonide, Tacrolimus (FK), Cyclosporine (CsA), Mycophenolic acid (MMF), Sirolimus, Anti-thymocyte globulin (ATG), or Alemtuzumab (Campath), prior receipt of one or more antivirals including Ganciclovir, Valganciclovir, Foscarnet, Cidofovir, Brincidofovir, and Rituximab, prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor, and prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir.

### BRIEF DESCRIPTION OF THE DRAWINGS

Various embodiments of this disclosure that are proposed as examples will be described in detail with reference to the following figures, wherein like numerals reference like elements, and wherein:

FIG. 1 is an overview of an exemplary computational approach for predicting efficacy of a therapeutic approach according to some embodiments of the present disclosure;

FIG. 2 shows the similarity of a VAE dataset generated by a generative model of the computational approach to an original dataset according to some embodiments of the present disclosure;

FIGS. 3A and 3B show Spearman correlations for the ACES dataset and the VAE-generated dataset, respectively;

FIGS. 4A, 4B and 4C show the distribution of the performance metrics for each dataset;

FIGS. 5A, 5B and 5C show precision-recall curves for each dataset;

FIG. 6 shows the contribution of all features included in a predictive model of the computational approach to the model's predicative ability;

FIG. 7 shows quality scores for all variables after synthesis by three methods;

FIGS. 8A, 8B, 8C and 8D show general trends in Spearman correlations for the ACES, VAE-generated, GAN-generated and GC-generated datasets, respectively;

FIGS. 9A, 9B and 9C show General trends in Spearman correlations for the ACES, Cincinnati, and Westmead datasets;

FIG. 10 shows that medium-sized VAE with medium or large number of nodes generates the data most similar to the real ACES records; and

FIGS. 11A, 11B, 11C, 11D, 11E, 11F and 11G show that custom neural network outperforms other hyperparameter-tuned classifier models when tested on withheld ACES records.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. Further, spatially relative terms, such as “top,” “bottom,” “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The order of discussion of the different steps as described herein has been presented for clarity sake. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

In order to choose the right treatments for individual patients, especially when considering VST, it is important to be able to accurately predict VST therapy clinical response. Artificial intelligence (AI), neural networks and AI models can be used to improve predictions and spot correlations in the date that might be useful in making predictions.

However, the small number of participants in many clinical trials limits the development of accurate models to predict treatment response. This is evidenced by the clinical trials of Virus-specific T-cell (VST) therapy to treat immunocompromised patients experiencing viral infections, with varying success rates (65-95%). Predictive models for VST response have been lacking due to the small number of participants (less than 100 on average) enrolled in these clinical trials. According to the present disclosure, a generative artificial intelligence (AI) approach is developed for predicting the likelihood of response to VST therapy.

VST therapy is a therapeutic approach to treat viral infections, which are a common cause of morbidity and mortality in immunocompromised patients, especially in patients with inborn errors of immunity, such as severe combined immunodeficiency (SCID), (as evidenced and disclosed by Gratwohl, A. et al. “Cause of death after allogeneic haematopoietic stem cell transplantation (HSCT) in early leukaemias: an EBMT analysis of lethal infectious complications and changes over calendar time.” Bone Marrow Transplant. 36(9), 757-769 (2005); Schladt, D. P., Israni, A. K. “Transplant rate OPTN/SRTR 2022 Annual Data Report: Introduction.” Am J Transplant. 24(2S1), S10-S18 (2024); and Dorsey, M., Puck, J. “Newborn Screening for Severe Combined Immunodeficiency in the US: Current Status and Approach to Management.” Int. J. Neonatal Screen. 3, 15 (2017), which are incorporated herein by reference in their entirety). Immunocompromised patients include those with transplants (>40K per year), those with cancer (>1M per year), those with general immunodeficiencies (˜106K in US), those with Adenovirus (AdV), Cytomegalovirus (CMV), or Epstein-Barr Virus (EBV).

VSTs are often considered second line or combination therapies which can be used with antivirals, Reduction of Immunosuppression (RI) and/or Monoclonal Antibodies (mABs).

In VST therapy development, healthy human T cells are isolated from peripheral blood mononuclear cells (PBMCs) by either selection or ex vivo expansion. They are infused into immune-compromised patients either prophylactically or for the treatment of active infections. VST infusion after first-line therapy with antivirals is effective in up to 95% of patients, with a range of 65-95% response in prior studies, with minimal risks of toxicity or graft-versus-host disease (GVHD), as evidenced by Bollard, C. M., Heslop, H. E. “T cells for viral infections after allogeneic hematopoietic stem cell transplant.” Blood. 127(26), 3331-3340 (2016); and Keller, M. D., Bollard, C. M. “Virus-specific T-cell therapies for patients with primary immune deficiency.” Blood. 135(9), 620-628 (2020), which are incorporated herein by reference in their entirety.

The reasons for the variable patient response rate are not fully understood. In studying the response rate to third-party VST products, multiple patients receiving the same VST formulation achieve different responses, indicating that the variation in response is unlikely to be entirely product-dependent. Instead, some patient characteristics will likely affect the response to VST therapy, including their comorbidities and prior treatments, as evidenced by Keller, M. D., Bollard, C. M. “Virus-specific T-cell therapies for patients with primary immune deficiency.” Blood. 135(9), 620-628 (2020). One clinical feature that is likely to affect VST treatment outcome is the use of immunosuppressive medication during VST therapy. Immunosuppression is known to impair response to VST therapy, and reduction of immunosuppression poses a greater risk to the patient, primarily transplant rejection. However, even with immunosuppressive medication, many patients still achieve viral clearance with VST therapy, and the primary cause of non-response to VST therapy remains unclear, as evidenced by Keller, M. D., Bollard, C. M. “Virus-specific T-cell therapies for patients with primary immune deficiency.” Blood. 135(9), 620-628 (2020). A major challenge in treating virally infected patients is to decide whether a patient should receive a VST infusion or another course of antiviral therapy with continued risks of toxicities and antiviral resistance. Because the underlying mechanism behind the varying responses is not well understood, the decision is complicated. A predictive model that can predict patient responses will be highly desired to identify meaningful clinical features for response and increase the success rate. Such a model has been lacking due to the “small n” problem in many clinical trial datasets.

In an embodiment, synthetic patient-response training data can be generated using a variational autoencoder (VAE), an expansion of a conventional AE, whose presence boosts the performance of predictive machine-learning models. The VAE can establish a mapping between input data (e.g., original clinical data) in an input space and a probability distribution across a latent space, and maps latent variables of the input space from the latent space to the input space to reconstruct the input data and obtain output data (e.g., synthetic data) that are in the form of variations of the input data. The distribution can be represented by the mean and variance of a Gaussian distribution, for example. The predictive model determines patient response with high accuracy, evidenced by cross-validation using data from three independent VST trials. The combined generative and predictive models provide a solution to selecting participants in VST clinical trials, and offer a generally applicable framework to enhance the performance of predictive models using small-scale clinical trial datasets.

According to the present disclosure, a solution to this “small n” problem in clinical trial datasets is proposed using a generative artificial intelligence (genAI) approach, and methodology is applied to three independent VST clinical trial cohorts. genAI models have demonstrated great success in natural language processing, imaging and video generation, as evidenced by Koohi-Moghadam M., Bae K. T. “Generative AI in Medical Imaging: Applications, Challenges, and Ethics.” J Med Syst. 47(1):94 (2023); Devlin, J., Chang, M., Lee, K., Toutanova, K., “BERT: Pre-training of Deep Bidirectional Transformers for Language Understanding.” Proceedings of NAACL-HLT, 4171-4186 (2019); Vaswani, A., et al., “Attention Is All You Need.” NIPS (2017); and Rombach, R., Blattmann, A., Lorenz, D., Esser, P., Ommer, B. “High-Resolution Image Synthesis with Latent Diffusion Models.” Proceedings of the IEEE/CVF conference on computer vision and pattern recognition (2022), which are incorporated herein by reference in their entirety. Most recently, genAI has been successfully applied to address the issue of limited training data in medical imaging modeling and, low-dimension, tabularized datasets, as evidenced by Kingma, D. P., Welling, M. “Auto-Encoding Variational Bayes.” ICLR conference. (2014); Chen, Y., Shi, F., Christodoulou, A. G., Xie, Y., Zhou, Z., Li, D. “Efficient and Accurate MRI Super-Resolution Using a Generative Adversarial Network and 3D Multi-level Densely Connected Network.” Proceedings of International Conference on Medical Image Computing and Computer Assisted Intervention (2018); and Hollmann, N., “Accurate predictions on small data with a tabular foundation model.” Nature. 637(8045), 319-326 (2025), which are incorporated herein by reference in their entirety. Despite these applications, the use of genAI to address the “small n” problem in clinical trial datasets has been lacking.

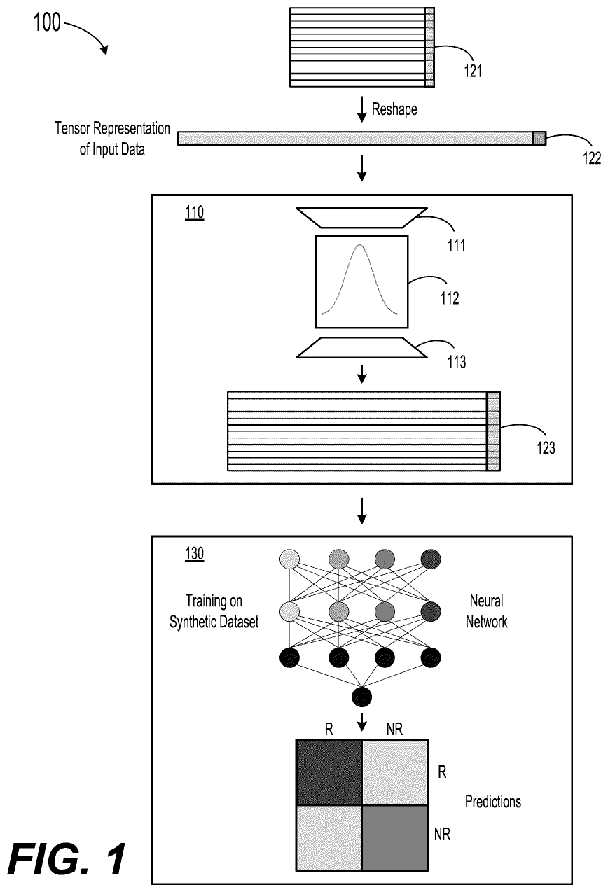

FIG. 1 is an overview of an exemplary computational approach 100 according to some embodiments of the present disclosure. Real original clinical records (or data or dataset) can be input to a variational autoencoder based method, which comprises or consists of encoding, sampling, and decoding to output synthetic records (or data or dataset). The synthetic records can then be used as input to a deep neural network (DNN), which is trained over, for example, 45 epochs. The DNN model can then be tested on withheld real clinical records to predict the likelihood of response and non-response using, for example, a classifier.

In an embodiment, the computational approach 100 can include a first artificial neural network 110, e.g., a variational autoencoder (VAE) 110, that can perform input reconstruction. For example, the VAE 110 can take input data (e.g., the original clinical data) and produce new data (e.g., synthetic data) that shares the same distributions as the original input data. The VAE 110 can include an encoder 111, a sampler 112 and a decoder 113. The encoder 111 can scale down the original data (e.g., clinical data or clinical dataset) 121 and compress and encode them through dimensionality reduction into a lower-dimensional representation in a latent space (e.g., tensor representation of input data). The sampler 112 can perform a random sampling from the latent space by establishing a mapping between the input data and a probability distribution across the latent space. For example, the probability distribution can be represented by the mean and variance of a Gaussian distribution. The decoder 113 can rescale the input data, reconstructing the dimensionality of the original data 121 to generating synthetic data 123, as disclosed by Kingma, D. P., Welling, M. “Auto-Encoding Variational Bayes.” ICLR conference. (2014). In the decoder 113, each subsequent layer contains a progressively larger number of active nodes. The synthetic data 123 generated by the VAE 110 can have a structure that is highly similar to the original data 121, and the performance of a machine learning model that predicts patient responses to VST can be boosted. In some embodiments, the encoder 111 and the decoder 113 can be both implemented using neural networks, with the aim of acquiring an optimal encoding-decoding scheme through an iterative optimization process such as gradient descent that can adjust model weights in a way that minimizes the difference between the original data input (i.e., the original data 121) the and the decoder's 113 output (i.e., the synthetic data 123). The predictive model, i.e., the computational approach 100, can be validated using data from two other independent VST cohorts, demonstrating its applicability across different cohorts.

Recently, there is an increasing interest in applying computational techniques originally designed for non-clinical problems to clinical studies. One study suggested the hypothetical use of a variational autoencoder for increasing sample sizes without having to increase recruitment in clinical studies, as disclosed by Papadopoulos, D., Karalis, V D. “Variational Autoencoders for Data Augmentation in Clinical Studies.” Appl. Sci. 13(15), 8793 (2023), which is incorporated herein by reference in its entirety. Deep learning models have been used to solve classification problems in the clinical space for decades. Multi-layer sequential models, comprising or consisting of dense layers with varying activation functions can be used to model the relationships between multi-variable inputs to make predictions, as disclosed by Vaswani, A., et al., “Attention Is All You Need.” NIPS (2017), which is incorporated herein by reference in its entirety. While these approaches are often used for highly dimensional data, and there are approximately 40 variables, they can also be used when the underlying relationships between the input variables are unknown or complex, as evidenced by Rombach, R., Blattmann, A., Lorenz, D., Esser, P., Ommer, B. “High-Resolution Image Synthesis with Latent Diffusion Models.” Proceedings of the IEEE/CVF conference on computer vision and pattern recognition (2022); and Chen, Y., Shi, F., Christodoulou, A. G., Xie, Y., Zhou, Z., Li, D. “Efficient and Accurate MRI Super-Resolution Using a Generative Adversarial Network and 3D Multi-level Densely Connected Network.” Proceedings of International Conference on Medical Image Computing and Computer Assisted Intervention (2018), which are incorporated herein by reference in their entirety. Given the lack of definitive data for specific variables and VST response, it is appropriate to utilize a deep learning approach for the classification of response and non-response. In turn, a variational autoencoder approach (e.g., the VAE 110) can be applied to increase the sample size from a VST therapy clinical study (e.g., the original data 121) to enable stronger statistical analyses and computational modeling results, as shown in FIG. 1.

### Results

a. Response Trends Across Datasets

In an embodiment, three VST clinical trial datasets, referred to as “ACES”, “Cincinnati”, and “Westmead”, are collected from three different cohorts (see DATA & METHODs). In ACEs, primary immunodeficiency disorder (PID) or stem cell transplant (HSCT) with an EBV, CMV or AdV infection are provided and patients have a persistent VMC, AdV or EBV infection after antiviral therapy, given partially-HLA matched allogeneic VSTs. Various data, such as type of Antivirals, steroids and immunosuppressives, infection type, viral loads (measured by PCR), and BMT donor type, and response are collected. Clinical response rates to VST infusions varied across the three datasets with 45% of ACES infusions, 78% of Cincinnati infusions, and 76% of Westmead infusions resulting in an antiviral response, based on defined protocol definitions: a minimum 1-log fold decrease in viral load at 28 days post-infusion, as measured via PCR (see Table, shown below).

TABLE
(Patient Demographics)

| | ACES | | Cincinnati | | Westmead | |
|---|---|---|---|---|---|---|
| Infusion Count | 79 | | 119 | | 49 | |
| Median Age (range) | 10.9 years (1 month- | | 15.2 years (1 month- | | 60 years (1-71 | |
| Underlying Diagnosis | 45 years) | | 73.6 years) | | years) | |
| Malignancy | 33 | (42%) | 53 | (45%) | 42 | (86%) |
| Inborn Error of | 26 | (33%) | 49 | (41%) | 7 | (14%) |
| Immunity | | | | | | |
| Other Non-Malignant | 20 | (25%) | 4 | (3%) | 0 | (0%) |
| Hematologic | | | | | | |
| Codition | | | | | | |

HSCT Donor Types

| | | | | | | |
|---|---|---|---|---|---|---|
| MMRD | 28 | (35%) | 24 | (20%) | 5 | (10%) |
| MMUD | 6 | (8%) | 27 | (23%) | 3 | (6%) |
| None | 5 | (6%) | 1 | (0.84%) | 0 | (0%) |
| UCBT | 11 | (14%) | 0 | (0%) | 0 | (0%) |
| MMRD + Cord | 3 | (4%) | 0 | (0%) | 0 | (0%) |
| MUD | 14 | (18%) | 40 | (34%) | 30 | (61%) |
| MRD | 9 | (11%) | 9 | (8%) | 10 | (20%) |
| abTCR depletion | 16 | (20%) | 4 | (3%) | 0 | (0%) |

Target Virus

| | | | | | | |
|---|---|---|---|---|---|---|
| CMV | 38 | (48%) | 60 | (50%) | 46 | (94%) |
| AdV | 43 | (54%) | 39 | (33%) | 1 | (2%) |
| EBV | 6 | (8%) | 37 | (31%) | 1 | (2%) |

Prep Category

| | | | | | | |
|---|---|---|---|---|---|---|
| RIC | 24 | (30%) | 24 | (20%) | 31 | (63%) |
| MAC | 45 | (57%) | 27 | (23%) | 18 | (37%) |
| None (no transplant) | 10 | (13%) | 62 | (52%) | 0 | (0%) |

Median Normalized Log Viral Load at

| | | | | | | |
|---|---|---|---|---|---|---|
| Infusion (range) | 3.72 | (0-6.69) | 3.39 | (0-7.04) | 3.51 | (2.17-5.59) |
| Response | 43 | (54%) | 96 | (81%) | 37 | (76%) |
| HLA Match | 4 | (1-6) | 2 | (0-6) | 2 | (1-4) |

Medications

| | | | | | | |
|---|---|---|---|---|---|---|
| Steroids | 23 | (29%) | 48 | (40%) | 10 | (20%) |
| Budesonide | 4 | (5%) | 5 | (4%) | 0 | (0%) |
| FK | 33 | (42%) | 38 | (32%) | 6 | (12%) |
| CsA | 6 | (8%) | 19 | (16%) | 24 | (29%) |
| MMF | 4 | (5%) | 6 | (5%) | 8 | (16%) |
| Sirolimus | 7 | (9%) | 6 | (5%) | 0 | (0%) |
| Gancidlovir | 19 | (24%) | 22 | (18%) | 18 | (37%) |
| Valganciclovir | 5 | (6%) | 9 | (8%) | 2 | (4%) |
| Foscarnet | 10 | (13%) | 19 | (16%) | 7 | (14%) |
| Cidofovir | 36 | (46%) | 14 | (12%) | 2 | (4%) |
| Brincidofovir | 9 | (11%) | 4 | (3%) | 0 | (0%) |
| Rituxan | 6 | (8%) | 0 | (0%) | 0 | (0%) |
| Acyclovir | 2 | (3%) | 0 | (0%) | 0 | (0%) |
| ATG | 30 | (38%) | 26 | (22%) | 0 | (0%) |
| Campath | 20 | (25%) | 31 | (26%) | 0 | (0%) |

The collected dataset has other feature details, such as thrombotic microangiopathy (TMA) (microscopic blood clots in capillaries and arteries (Y/N)), veno-occlusive disease (VOD) (blockage of veins in the liver (Y/N)), graft vs host disease (GVHD) (none, low-grade, high-grade), adverse events (AE) (any other AE (could be multiple)), diagnostic category (IEI, malignant or non-malignant HC), prep category (myeloablative, reduced conditioning, none), BMT donor (matched related, matched unrelated, mismatched unrelated, mismatched related, cord), and abTCR CD19 (if abTCR/CD19 depleted transplant was given). The clinical problem is how to determine if a patient should continue with antiviral medication alone or if he should receive cellular therapy.

The correlations between individual variables and response in each of the three datasets are assessed. The variables with the strongest positive correlations with response in the ACES dataset were BMT Donor Type Matched Related (0.25), Campath (0.24), Acyclovir (0.15), Original diagnosis of Malignancy (0.11), and Mycophenolate mofetil (MMF) (0.1). In the Cincinnati dataset, the top positive correlators with response were Cyclosporine (CsA) (0.21), MMF (0.11), Sirolimus (0.11), Budesonide (0.10), and EBV infection (0.10). In the Westmead dataset, the top positive correlators with response were Foscarnet (0.23), FK (0.21), MMF (0.12), Valganciclovir (0.12), and Cidofovir (0.12). Interestingly, receiving MMF as an immunosuppressive agent has a small positive correlation with response in all three datasets.

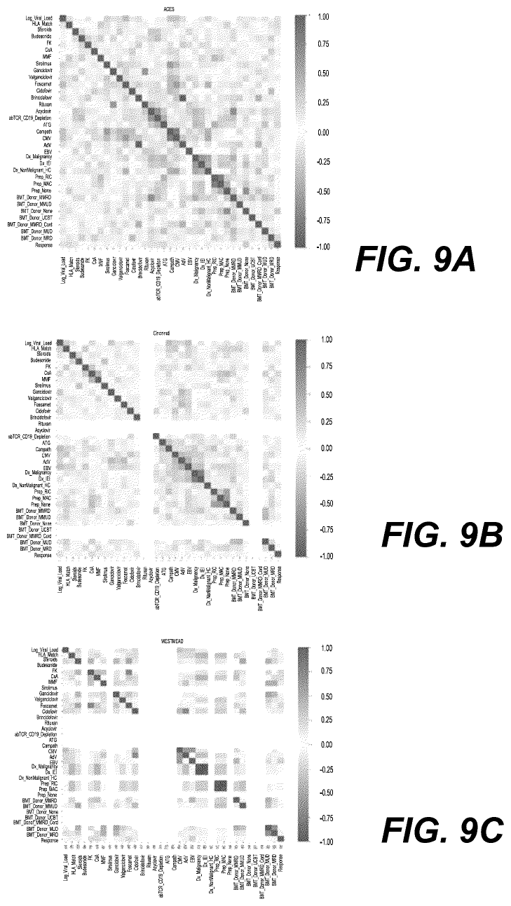

The strongest negative correlators with response in the ACES dataset were not having a bone marrow transplant (−0.28), Steroids (−0.20), ATG (−0.17), abTCR CD19 depletion (−0.17), and the normalized log viral load at infusion (−0.16). Top negative correlators with response in the Cincinnati dataset were log normalized viral load at infusion (−0.18), Original diagnosis of non-malignant hematologic condition (−0.14), Brincidofovir (−0.14), ATG (−0.10), Campath (−0.10), and Ganciclovir (−0.10). Top negative correlators with response in the Westmead dataset were having an EBV infection (−0.25), CsA (−0.20), Steroids (−0.06), BMT Donor type matched related (−0.06), and Ganciclovir (−0.06). The negative correlations with response drop off in strength much more quickly in the Westmead (adult) dataset than in the two pediatric datasets. Of note, the log normalized viral load has a negative correlation with response in all three datasets (correlation of −0.04 in the Westmead dataset), indicating that as the viral load at the time of infusion increases the likelihood of response to VST therapy decreases, as shown in FIGS. 9A-9C. FIGS. 9A-9C show General trends in Spearman correlations for the ACES, Cincinnati, and Westmead datasets.

### B. Generation of Synthetic VST Infusion Records

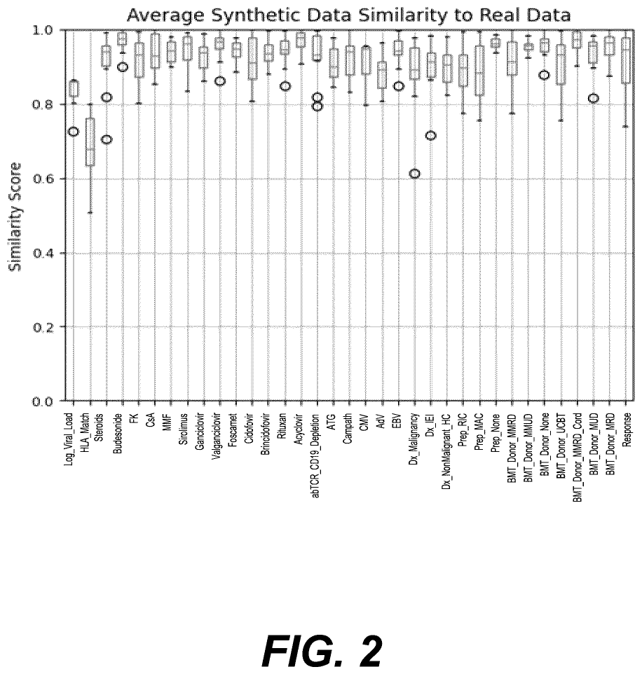

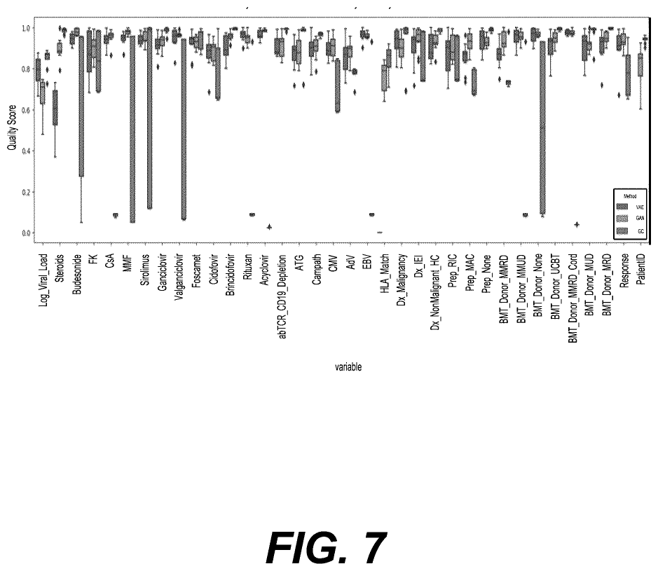

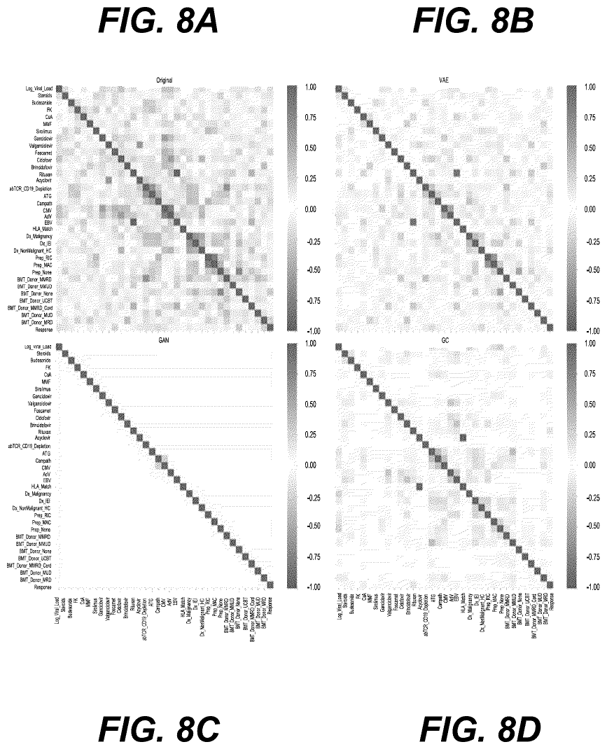

To overcome the limitations in predictive power due to small input sample size, generative methods (e.g., using the VAE 110) are first employed to increase the sample size for a downstream classifier 130, as shown in FIG. 1. In an embodiment, the VAE 110 is combined with biological rules to synthesize VST infusion data from a small, real clinical dataset of limited size, and the synthesized VST infusion data can be utilized to train a deep learning model to predict the likelihood of response. Several methods for data synthesis are tested, most notably a VAE, a generative adversarial network (GAN), and a gaussian copula synthesizer (GC), as disclosed by Devlin, J., Chang, M., Lee, K., Toutanova, K., “BERT: Pre-training of Deep Bidirectional Transformers for Language Understanding.” Proceedings of NAACL-HLT, 4171-4186 (2019); Yang, S., Zhu, F., Ling, X., Liu, Q., Zhao, P. “Intelligent Health Care: Applications of Deep Learning in Computational Medicine.” Front. Genet. 12 (2021); and Dufera, T. T. “Deep neural network for system of ordinary differential equations: Vectorized algorithm and simulation.” Machine Learning with Applications. 5 (2021), which are incorporated herein by reference in their entirety. To train and evaluate the three methods, the ACES dataset was split into training and testing groups using 10-fold cross validation. After data splitting and preparation, 59 pre-VST infusion records from the ACES cohort were used as the input to each of these models and 5,000 synthetic VST infusion records were generated. After data generation, the synthetic datasets' similarities to the real VST infusion dataset (ACES dataset) are accessed, using the Total Variation Distance complement, Kolmogorov-Smirnov complement, and Spearman correlations. The VAE 110 can generate the synthetic data 123 with high quality with the subset of ACES records as the input data 121. For continuous variables, quality can be measured as the KS complement. For binary variables, quality can be measured as the TV complement. As shown in FIG. 2, the VAE generated dataset is highly similar to the original dataset, where on average, each individual synthetic variable's distribution is over 90% similar in terms of distribution to the original distribution. In comparing the inter-variable relationships, the VAE is best able to reproduce correlations seen in the ACES dataset. The GAN and GC models are unable to produce data which share the same correlative patterns as the real data, as shown in FIGS. 7 and 8A-8D. The VAE generated data have intervariable correlations most similar to those in the real data when compared to the GAN and GC. VAEs and GANs are both based on neural network, which may be a better fit for our data than the GC structure. However, GANs can be more complicated to train, which may be why the VAE appears to more successfully generate clinical records when compared to the GAN, as evidenced by Bacigalupo, A., Boyd, A., Slipper, J., Curtis, J., & Clissold, S. “Foscarnet in the management of cytomegalovirus infections in hematopoietic stem cell transplant patients.” Expert review of anti-infective therapy, 10(11), 1249-1264 (2012), which is incorporated herein by reference in its entirety.

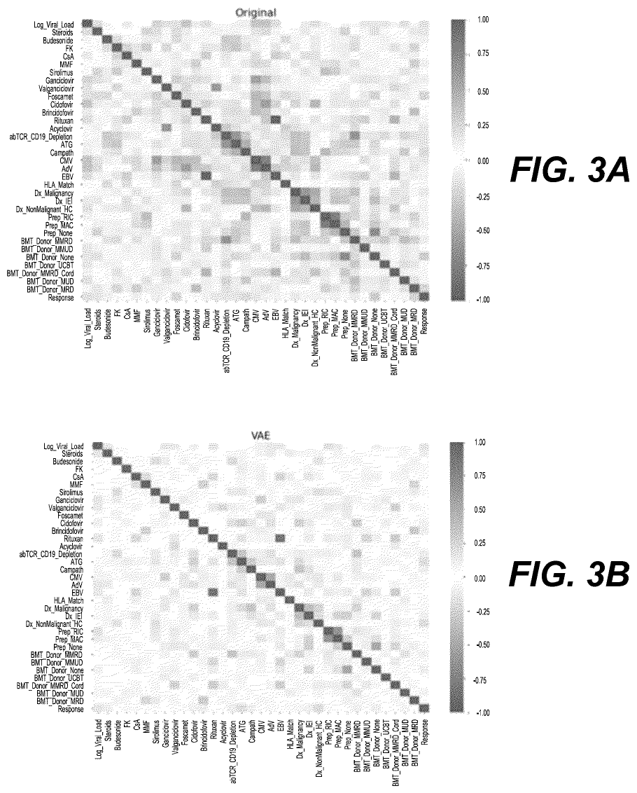

Next, the relationships between variables are compared to confirm if these are maintained in synthetic data. From the quality scores alone, it is difficult to determine which method is the best approach. All three methods handle the binary and categorical variables well, with the VAE and GAN slightly outperforming the GC. For all categorical and binary variables, the VAE and GAN both achieve over 80% quality, whereas some variables for the GC are under 80%, as shown in FIGS. 3A-3B and 8A-8D. FIGS. 3A-3B show Spearman correlations for the ACES dataset and the VAE-generated dataset, respectively. FIGS. 8A-8D show general trends in Spearman correlations for the ACES, VAE-generated, GAN-generated and GC-generated datasets, respectively. All three methods struggle to accurately generate the viral load data, likely due to the fact that there is variation in viral load measurements from one institution to another, creating additional intravariable variation that may be difficult to replicate accurately. Next, the relationships between variables are considered. Because these data will be used to predict response, it is important that the generative method is able to maintain relationships between variables. VAE synthesized data maintains both the intervariable relationships seen in the real data as measured by Spearman correlations and the individual variable distributions, indicating their use in overcoming the small sample size problem.

After generation, the majority of VAE-synthesized data are usable and represent realistic patients, with on average only 1.5% of generated records breaking biological rules, i.e., if a single record contains true values for both Campath and ATG. The synthetic data were highly similar to the real ACES data, with an average similarity score of over 0.9. However, approximately 72% of records are kept after checking all biological requirements.

### C. Optimization of the VAE Structure

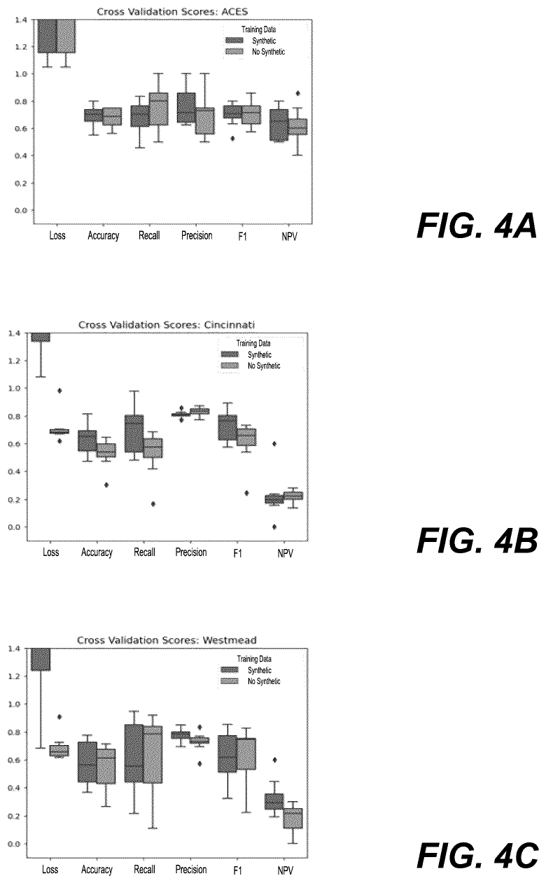

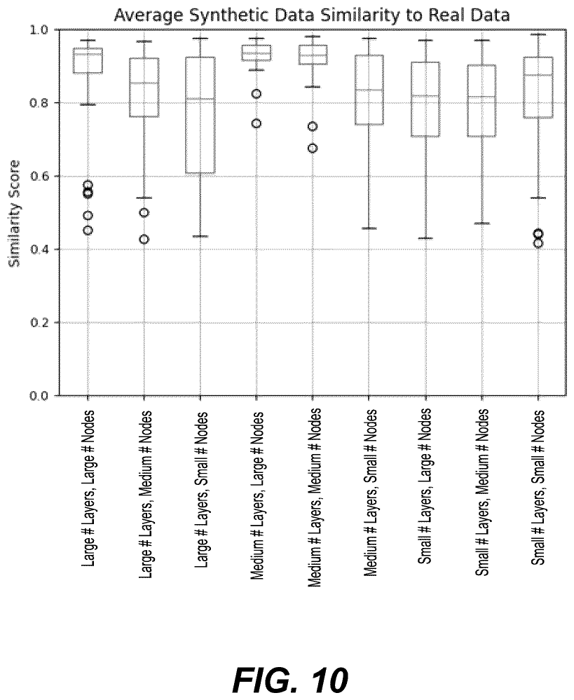

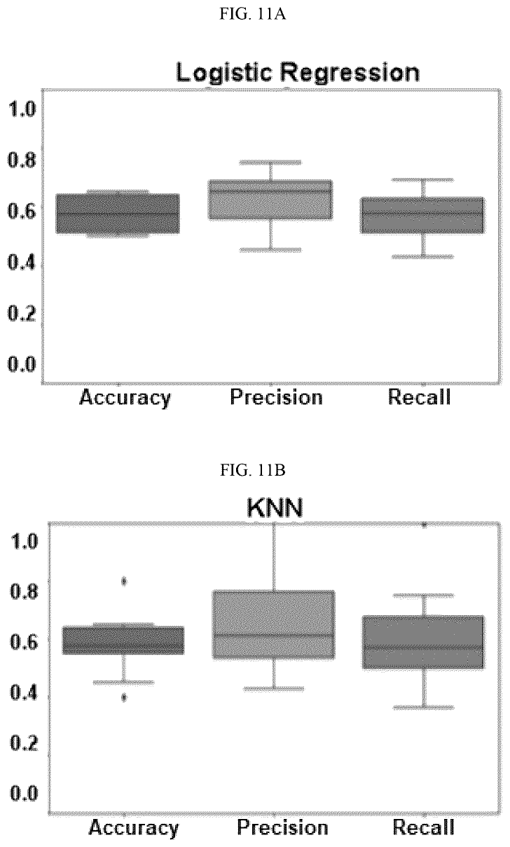

After determining the VAE to be the most suitable generative method for our data, the optimal VAE structure can be determined by changing the number of layers and number of nodes. To do so, 9 combinations of varying number of layers and nodes are tested. To assess the performance of each model, the similarity scores were averaged across all variables to get a single value. All 9 models had median quality scores of 0.8 or higher, likely due to the nature of generating binary variables. However, the distribution of the model scores varies, where the model with the fewest number of layers has the largest variation (nearly 0.5). The NN classifier 130 can accurately predict the likelihood of response on internal and external datasets. FIGS. 4A-4C show that the distribution of the performance metrics for each dataset with 10-fold cross validation, including loss, accuracy, recall, precision, and Negative Predictive Value (NPV). Loss is measured as the binary cross entropy and NPV is measured as True Negatives/(True Negatives+False Negatives). The models with more layers and nodes have smaller variations than the small models; however, they also had several outliers with similarity scores below 0.6. The best performing models are those with a medium number of layers and large or medium number of nodes. Both had median similarity scores over 0.9 and lower quartiles over 0.8, with only 2 outliers over 0.6, as shown in FIG. 10. FIG. 10 shows that medium-sized VAE with medium or large number of nodes generates the data most similar to the real ACES records. The model with medium number of layers and nodes are selected, because the increase in performance between the medium and large models was minimal.

### D. Prediction of VST Therapy Clinical Response

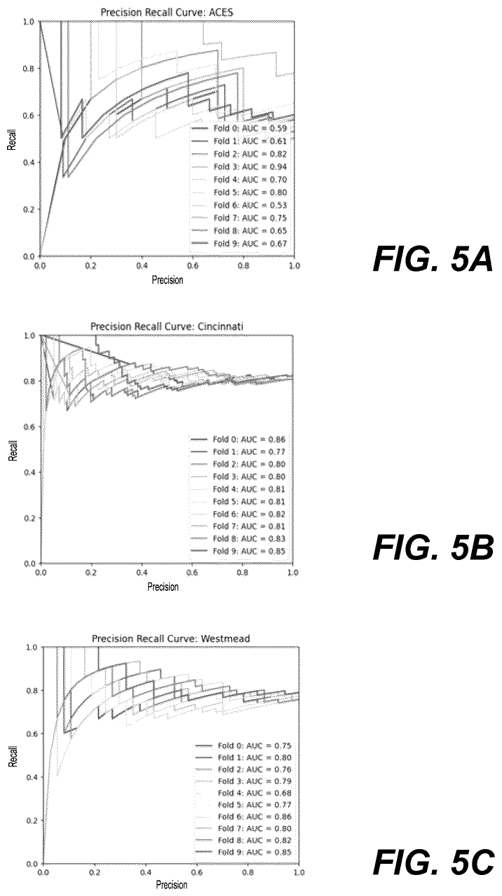

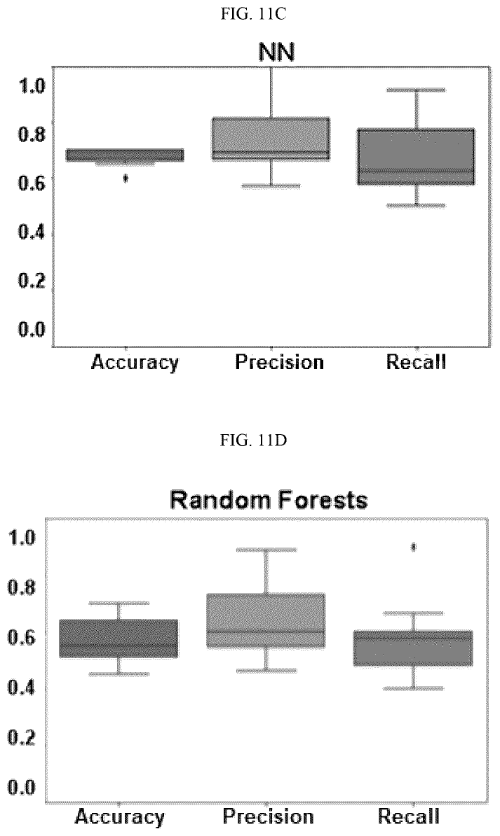

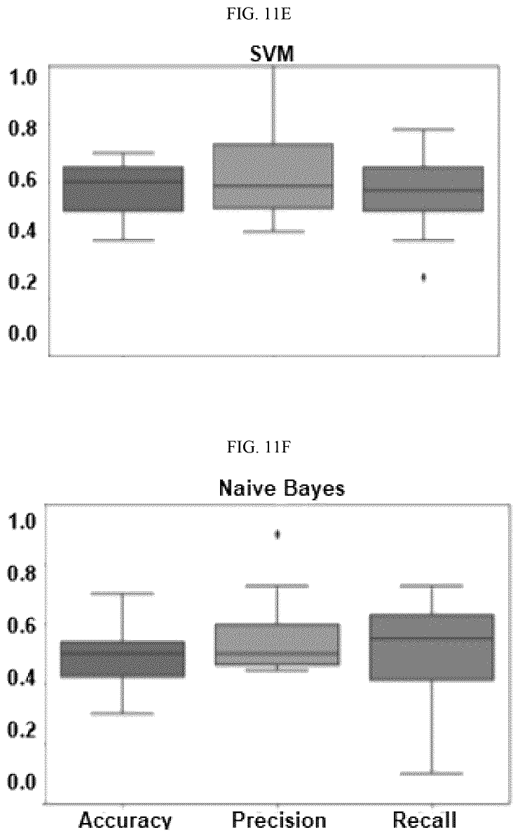

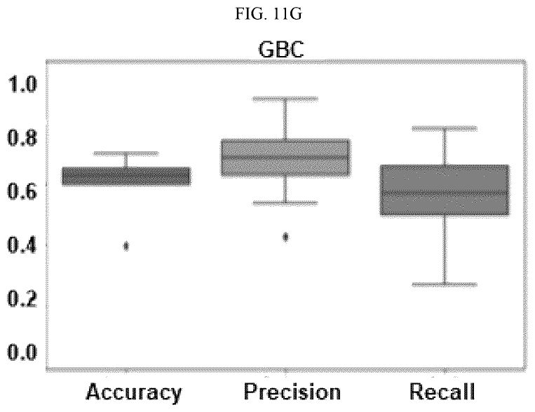

After sufficient high quality synthetic pre-infusion data are generated, the predictive classification problem is implemented. Several standard classifier methods, a logistic regression (LR) model, naïve Bayes (NB) model, and a support vector machine (SVM), as well as custom neural network (NN), as tested. With an input of 35 pre-VST infusion variables, each classifier can be trained to predict the likelihood of response or non-response to VST therapy. After training on ˜5,000 synthetic records over 45 epochs, the models on withheld ACES records are tested. The subset of the ACES dataset used for testing was not seen by the VAE or by the classifiers prior to testing. Our custom NN outperformed other models, achieving over 70% accuracy, recall, precision, and F1-scores when tested on the withheld data. FIGS. 5A-5C show that neural network classifier accurately predicts the likelihood of patient response to VST therapy when tested on unseen, real patient data from 3 cohorts (A) ACES, (B) Cincinnati, (C) Westmead. The other classifiers all scored below median 80% accuracy, with varying precision and recalls, as shown in FIGS. 11A-11G. FIGS. 11A-11G show that custom neural network outperforms other hyperparameter-tuned classifier models when tested on withheld ACES records.

To further assess the model's robustness, the NN classifier's performance on independent datasets, not seen by any part of the model, are tested. Of note, most records in the Cincinnati and Westmead cohorts are responders. When class imbalance occurs, a more popular metric is the precision-recall curve, which is included and shows promising results for these datasets. The Cincinnati and Westmead datasets contain the same input variables but were not used for any of the prior modeling experiments. The classifier achieves median over 60% accuracy, recall, precision, and f1-scores on both datasets. The classifier's ability to accurately predict the likelihood of response in multiple unseen datasets indicates its viability.

### E. Feature Importance

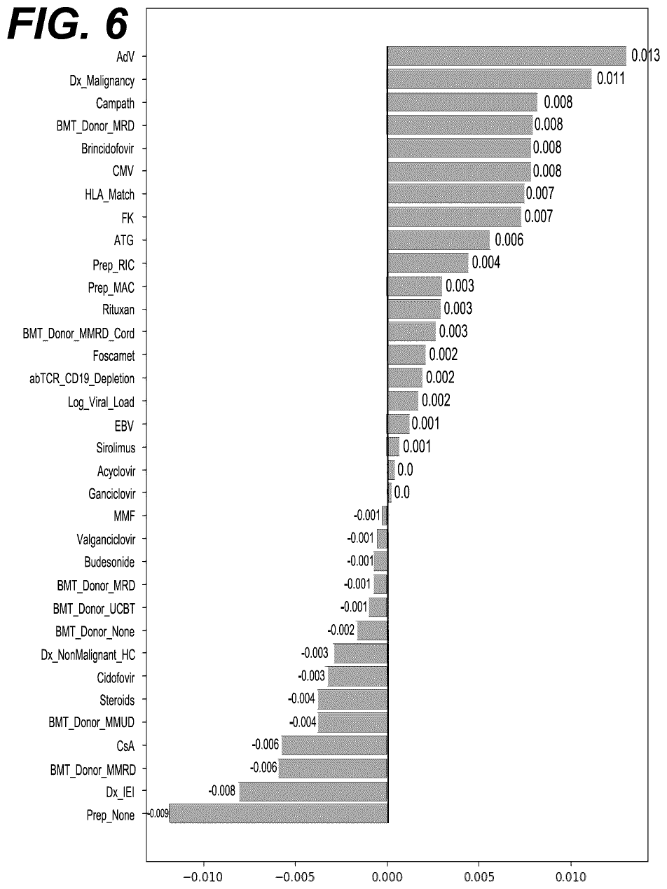

In the assessment of the best performing model, the NN, the feature importance is also considered, with the goal of identifying if any individual variables were contributing most to the model's predictive ability. The top positive contributors are identified as CMV, BMT donor type mismatched unrelated, no prep category for BMT (i.e., no known BMT and/or no known prep type), BMT donor type mismatched related and cord blood, and BMT donor type matched unrelated donor. While none of these variables had a strong correlation with the response in the clinical datasets using Spearman values, the Shapley values indicate their value in patient stratification, where MMF, for example, has a positive predictive value and small positive correlation with response in the ACES dataset, as shown in FIG. 6. FIG. 6 shows that all features included in the NN classifier 130 contribute to the model's predicative ability, with the top positive contributing feature as if a patient has a CMV infection or not and the top negative contributing feature as whether or not a patient received ATG. Feature importance is measured as the Shapley values, where larger values indicate the feature contributes more to the model's predictive ability. Negative values indicate the model being more likely to predict a 0 or non-response and positive values indicate the model being more likely to predict a 1 or response, i.e., as the viral load increases the likelihood of predicting a response decreases. FIG. 6 shows that the values are all small, indicating that each feature has a relatively small individual contributions, so the model relies more on the combination of features that are generated by the VAE 110.

### Discussion

Choosing the right treatments for individual patients is critical to maximize the treatment effect. In VST therapy, the decision of selecting patients for VSTs versus continuing with additional course(s) of antiviral therapy is particularly challenging. The lack of large datasets, complex underlying relationships between clinical variables, and variations in how response may be determined, all contribute to the difficult nature of this problem. According to the present disclosure, a novel dual-modeling framework is presented, where genAI (e.g., the VAE 110) was first used to generate accurate synthetic clinical data, followed by training a classification model (e.g., the downstream classifier 130) to predict patient response using these synthetic data points. It is shown that a VAE approach can generate data which are highly similar to real data in terms of individual variable distributions and inter-variable relationships (e.g., mutual exclusiveness). A machine learning model can then be trained on the synthetic data to accurately predict the likelihood of therapeutic response.

In an embodiment, the classifier 130 can include a neural network (NN) model, e.g., a convolutional NN (CNN), that may comprise or consist of hundreds of layers and millions of parameters, e.g., weights, biases, kernels and activation function, and involve complex vector and matrix computations at each layer. However, too large a CNN model may be too complex to be efficiently run on general hardware platforms. CNN model developers write algorithms with mathematical operations (or descriptions) supported by the API to describe their DL models. The API selects and trains a DL model (Tensorflow model), and converts the Tensorflow model using a converter to a Tensorflow Lite model (Tensorflow Lite flat buffer file (.tflite)), which can then be optimized, e.g., through quantization, which converts 32-bit floating points into 16-bit floating points or 8-bit floating points or integers, and weight pruning, which trims parameters within a model that have very less impact on the performance of the model. The optimized Tensorflow Lite model, after compiled by a compiler (e.g., TFLite interpreter) implemented in an edge device into machines codes (or instructions), can then be deployed on edge devices.

Foscarnet has the top positive correlation with response in the Westmead dataset, a primarily adult group, but has a small negative correlation with response in the ACES primarily pediatric dataset, and the Cincinnati mixed-age dataset, indicating a potential area of further investigation to the combined relationship between age, Foscarnet, and VST response. Prior studies of giving Foscarnet for CMV infections after transplant have not focused on the role of age, where most studies either include only adults or only children. However, Foscarnet has been shown to be effective in both adults and children, as evidenced by Hollmann, N., “Accurate predictions on small data with a tabular foundation model.” Nature. 637(8045), 319-326 (2025); Zhong, Y., Zhang, X., Zhou, L., Li, L., Zhang, T. “Updated analysis of pediatric clinical studies registered in ClinicalTrials.gov”, 2008-2019. BMC Pediatrics. 21, 212 (2021); and LeCun Y., Bengio Y., Hinton G., “Deep learning.” Nature. 28; 521(7553):436-44 (2015), which are incorporated herein by reference in their entirety.

Future directions to improve the accuracy of the approach according to the present disclosure would include training on more datasets to improve the model's performance across different cohorts. The F1 score of −70% implies a use case of stratifying patients in a future trial to split them into candidates for VST versus secondary antiviral treatment, which could potentially increase response rates for VST therapy by avoiding administration of VSTs to patients who are unlikely to benefit. The model disclosed according to the present disclosure could consider more features including the specific VST products and/or selection criteria, which undoubtedly contributes to response rates. Computational modeling, including genAI approaches, have been proposed for treatment selection to improve clinical outcomes, as evidenced by Shereck, E. B., Cooney, E., van de Ven, C., Della-Lotta, P., & Cairo, M. S. “A pilot phase II study of alternate day ganciclovir and foscarnet in preventing cytomegalovirus (CMV) infections in at-risk pediatric and adolescent allogeneic stem cell transplant recipients.” Pediatric blood and cancer, 49(3), 306-312 (2007); and Zhu, J., et al. “Comparison of valganciclovir versus foscarnet for the treatment of cytomegalovirus viremia in adult acute leukemia patients after allogeneic hematopoietic cell transplantation.” Leukemia & lymphoma, 65(6), 816-824 (2024), which are incorporated herein by reference in their entirety. The approach provided according to the present disclosure provides a promising solution to predict treatment responses of immunocompromised patients with viral infections.

In summary, it has been shown that a combined generative and classification approach can be used to accurately generate and predict patient clinical response after VST infusion. Synthetic data generated preserved correlations between features, and improved the performance of machine learning models to predict patient response to VST therapy. The classifier model provided according to the present disclosure can accurately predict patient response, achieving an average f1-score over 70% on the withheld ACES cohorts. It is identified that top clinical features contributing to the model's predictive power and confirmed their value in the VST setting. The method presented according to the present disclosure is a novel approach to patient stratification, which will enable better patient selection for VST therapy versus continued antiviral use. The framework disclosed herein will be generally applicable to building accurate predictive models for patient response, using clinical trial datasets with limited sample sizes.

### Data & Methods

a. Data

Metadata from the ACES clinical trial (NCT03475212) as well as the CHAPS trial (NCT, which included 79 pediatric VST infusions during years 2017-2020 are used for the primary modeling experiments (“ACES dataset”), as disclosed by Aggarwal, Alankrita & Mittal, Mamta & Battineni, Gopi. “Generative adversarial network: An overview of theory and applications.” International Journal of Information Management. 100004 (2021), which is incorporated herein by reference in its entirety. Patient age ranges from 1 month to 45 years, with a median age of 10.9 years. Two additional VST infusion datasets are used for testing, the Cincinnati Children's Hospital cohort (i.e., “Cincinnati dataset”) and the Westmead Hospital cohort (“Westmead dataset”), as disclosed by Hughes, J. “A unified Gaussian copula methodology for spatial regression analysis.” Sci Rep. 12(1):15915 (2022), which is incorporated herein by reference in its entirety. The Cincinnati dataset includes 199 VST infusion records, with patients ranging in age from 1 month to 73.6 years, with a median age of 15.2 years. The Westmead dataset contains 49 VST infusion records, with patients ranging in age from 1 year to 71 years, with a median age of 60 years.

The variables included in the dataset are continuous, categorical, or Boolean valued. Categorical data are one-hot encoded prior to modeling. Continuous variables are log normalized. Variables include: type of viral infection (i.e., AdV, EBV, CMV), viral load at time of infusion, original diagnosis (i.e., inborn error of immunity, malignant, non-malignant hematology condition), preparatory category (i.e., myeloablative, reduced conditioning, none), transplant donor (i.e., Mismatched related donor, Matched related donor, Matched unrelated donor, umbilical cord transplant, none), cellular depletion (i.e., abTCR, CD19), and receipt of immunosuppressive medications, including systemic corticosteroids, Budesonide, Tacrolimus (FK), Cyclosporine (CsA), Mycophenolic acid (MMF), Sirolimus, Anti-thymocyte globulin (ATG), Alemtuzumab (Campath), use of antivirals including Ganciclovir, Valganciclovir, Foscarnet, Cidofovir, Brincidofovir, Rituximab, and degree of HLA match between the patient and VST product. Degree of HLA match ranges from 1 to 6, based on the number of major alleles shared. Viral load is measured by PCR in IU/ml with relevant conversions for each virus type.

### B. Data Synthesis (VAE)

All models were built in Python 3.7, version information for relevant packages included in the supplementary information.

Of the 79 ACES records, 59 are converted to tensors and used as input to the VAE model. All of the 79 ACES recording are converted to a tensor representation but only 59 of 79 are used to train the VAE. The VAE model generates 5,000 synthetic records over 1000 training epochs. After generation, data are subject to removal if they do not meet biological requirements. For example, if a synthesized record returns 1 (true) for ATG and 1 (true) for Campath, it is removed from the synthetic dataset. This is because a real patient is highly unlikely to be administered both drugs concurrently. The model is cross validated; this process is repeated 10 times, with a random selection of the ACES cohort being utilized as input to the VAE model each time (and a corresponding selection of the ACES cohort being withheld for later testing). Thus, 10 synthetic datasets of approximately 5,000 records each are generated, with 10 corresponding sets of 20 withheld ACES records.

To assess synthetic data quality, similarity scores are calculated, comparing the distribution of a single variable in the ACES cohort to the distribution of that same variable in the synthetic cohort. For continuous variables, the Kolmogorov-Smirnov complement, as disclosed by Marco, R., Ahmad, S. S. S., Ahmad, S., “Improving Conditional Variational Autoencoder with Resampling Strategies for Regression Synthetic Project Generation.” Int J of Intelligent Engineering & Systems, 372-388 (2023), which is incorporated herein by reference in its entirety. For binary variables, the total variation distance complement is used as the similarity score, as disclosed by Dinstag, G. “Clinically oriented prediction of patient response to targeted and immunotherapies from the tumor transcriptome.” Med, 4(1), 15-30 (2023), which is incorporated herein by reference in its entirety. In both cases, higher similarity scores indicate more similar distributions, i.e., higher quality synthetic variables. Similarity scores are displayed as box-and-whisker plots for each of the ten datasets, as shown in FIG. 7. FIG. 7 shows box-and-whisker plots of similarity scores for individual variables, comparing the distributions in the ACES dataset to the synthetic dataset for three generative methods: VAE, GAN and GC. To assess inter-variable relationships, the Spearman correlation between each variable is calculated in each dataset and the average results are displayed in a heatmap, as disclosed by Kozlowska, E., Haltia, U. M., Puszynski, K., & Farkkila, A. “Mathematical modeling framework enhances clinical trial design for maintenance treatment in oncology.” Scientific reports, 14(1), 29721 (2024), which is incorporated herein by reference in its entirety.

### C. Classification

For the purposes of this model, response is encoded as 0 for non-response, or 1 for response. Responses included both partial and complete, where any notable drop in viral load after infusion is deemed a response. The final classifier, NN, comprises or consists of a convolutional layer with a kernel size of 4, a dense layer with a reLU activation, a dropout rate of 0.2, and final dense layer with sigmoid activation. The classification model is trained on the synthetic data, for 50 epochs, and the model is tested on the corresponding withheld real ACES records. The process is repeated for all 10 synthetic datasets, generating average results across each dataset. To assess the deep classifier's performance, AUC, accuracy, recall, and precision are calculated. Results are compared across the 7 classifiers, with tuning of relevant hyperparameters for each classifier, i.e., tuning the tolerance for the LR model.

The same methods of testing are used for assessment of the best model, the NN, on the Cincinnati and Westmead datasets.

### D. Feature Importance

In addition to the model's performance, feature importance was also assessed. The Shapley factors for feature contributions are used, where a larger Shapley value indicates greater predictive quality, as disclosed by Shapley, L. “A Value for n-Person Games.” In: Kuhn H. and Tucker, A., Eds., Contributions to the Theory of Games II. 307-317 (1953); and Chen, H., Lundberg, S M., Lee, S. “Explaining a series of models by propagating Shapley values.” Nat Commun. 13, 4512 (2022), which are incorporated herein by reference in their entirety.

In the preceding description, specific details have been set forth, such as a particular geometry of a processing system and descriptions of various components and processes used therein. It should be understood, however, that techniques herein may be practiced in other embodiments that depart from these specific details, and that such details are for purposes of explanation and not limitation. Embodiments disclosed herein have been described with reference to the accompanying drawings. Similarly, for purposes of explanation, specific numbers, materials, and configurations have been set forth in order to provide a thorough understanding. Nevertheless, embodiments may be practiced without such specific details. Components having substantially the same functional constructions are denoted by like reference characters, and thus any redundant descriptions may be omitted.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

“Substrate” or “target substrate” as used herein generically refers to an object being processed in accordance with the present disclosure. The substrate may include any material portion or structure of a device, particularly a semiconductor or other electronics device, and may, for example, be a base substrate structure, such as a semiconductor wafer, reticle, or a dielectric layer on or overlying a base substrate structure such as a thin film. Thus, substrate is not limited to any particular base structure, underlying dielectric layer or overlying dielectric layer, patterned or un-patterned, but rather, is contemplated to include any such dielectric layer or base structure, and any combination of dielectric layers and/or base structures. The description may reference particular types of substrates, but this is for illustrative purposes only.

Those skilled in the art will also understand that there can be many variations made to the operations of the techniques explained above while still achieving the same objectives of the present disclosure. Such variations are intended to be covered by the scope of this disclosure. As such, the foregoing descriptions of embodiments of the invention are not intended to be limiting. Rather, any limitations to embodiments of the invention are presented in the following claims.

What is claimed is:

1. A method for treating an immunocompromised patient comprising:
(a) collecting from the patient values for one or more variables, comprising selecting prior cancer remission or relapse, prior reaction after transplant, a degree of HLA match, type of viral infection, type of comorbidity or infection, viral load, prior receipt of one or more immunosuppressive medications, prior receipt of one or more anti-cancer medications, or prior receipt of one or more antiviral medications;
(b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and
(c) administering an antiviral drug to a patient who has a threshold score indicative of a likelihood of a therapeutic response to anti-viral therapy; and/or
administering VSTs to a patient who has a threshold score indicative of a likelihood of a therapeutic response to VST therapy.

2. The method for treating an immunocompromised patient according to claim 1 further comprising:
(a) collecting from patient values for one or more variables selected from the group comprising:
transplant donor and recipient age and sex,
presence or absence of inborn error of immunity, malignant, non-malignant hematology condition, or other primary immunodeficiency, upon original diagnosis,
presence or absence of partial or complete cancer remission including no detectable cancer, reduction or growth of a tumor, higher or lower number of cancer cells compared to prior levels, and symptomatic improvement or regression compared to a prior level,
prior graft-vs-host reaction after transplant,
presence or absence of myeloablative conditioning regiment (MA), reduced intensity conditioning regimen RIC), or no conditioning regiment (NMA),
transplant donor type including mismatched related donor, matched related donor, matched unrelated donor, umbilical cord cell transplant, or no donor,
a degree of HLA match ranging from 1 to 6 based on the number of major alleles shared, wherein said major alleles include HLA-A, HLA-B, HLA-C and HLA-DR, HLA-DQ and HLA-DP,
cellular depletion or ablation of TCRαβ, CD19, naive T cells (CD45RA+ T cells) and/or CD34+ T cells,
a level of CD8+ or CD8+ T cells or a ratio of CD4+ cells to CD8+ T cells or a higher or lower level compared to a prior level,
type of viral infection comprising adenovirus (AdV), Epstien-Barr Virus (EBV), Cytomegalovirus (CMV), Herpes Simplex Virus (HSV), human herpes virus 8, Varicella-Zoster virus, or human papillomarvirus,
type of comorbidity or infection caused by an opportunistic virus, bacterium, fungi, or parasite,
viral load at a time of infusion of antiviral drug or VST, wherein viral load can be measured in IU/ml by PCR,
prior receipt of one or more immunosuppressive medications comprising systemic corticosteroids, Budesonide, Tacrolimus (FK), Cyclosporine (CsA), Mycophenolic acid (MMF), Sirolimus, Anti-thymocyte globulin (ATG), Alemtuzumab (Campath), antivirals including Ganciclovir, Valganciclovir, Foscarnet, Cidofovir, Brincidofovir, or Rituximab,
prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor,
prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir;
(b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and
(c) administering an antiviral drug to a patient who has a threshold score indicative of a likelihood of a therapeutic response to anti-viral therapy; and/or
administering VSTs to a patient who has a threshold score indicative of a likelihood of a therapeutic response to VST therapy.

3. The method of claim 1, wherein the immunocompromised patient is infected by an opportunistic virus and is administered an antiviral drug and/or VST.

4. The method of claim 1, wherein the immunocompromised patient is infected by cytomegalovirus, Epstein-Barr virus, or Adenovirus; and wherein the patient is administered Ganciclovir, Valganciclovir, Foscarnet, Cidofovir, Brincidofovir, Acyclovir and Rituximab and/or administered VSTs that recognize Cytomegalovirus, Epstein-Barr virus, and/or Adenovirus.

5. The method of claim 1, wherein the immunocompromised patient has undergone a autograft, an allograft, or a xenograft and is infected by an opportunistic virus and is administered an antiviral drug and/or VST.

6. The method of claim 1, wherein the immunocompromised patient has undergone a autograft, an allograft, or a xenograft and is infected by cytomegalovirus, Epstein-Barr virus, or Adenovirus; wherein the patient is administered Ganciclovir, Valganciclovir, Foscarnet, Cidofovir, Brincidofovir, Acyclovir and Rituximab and/or administered VSTs that recognize cytomegalovirus, Epstein-Barr virus, and/or Adenovirus.

7. The method of claim 6, wherein the autograft, allograft or xenograft is bone marrow cells or stem cells.

8. The method of claim 1, wherein the immunocompromised patient has undergone a autograft, an allograft, or a xenograft, has been administered an immunosuppressant, and is infected by cytomegalovirus, Epstein-Barr virus, or Adenovirus; wherein the patient is administered Ganciclovir, Valganciclovir, Foscarnet, Cidofovir, Brincidofovir, Acyclovir and Rituximab and/or administered VSTs that recognize cytomegalovirus, Epstein-Barr virus, and/or Adenovirus.

9. The method of claim 8, wherein the immunosuppressant comprises Budesonide GI, Tacrolimus (FK), Mycophenolate mofetil (MMF), Sirolimus, Infliximad, Vedolizumad, Anti-thymocyte globulin (ATG) and Alemtuzumab (Campath).

10. The method of claim 1, wherein the patient has a primary or secondary immunodeficiency, is infected by an opportunistic virus, and is administered an antiviral drug and/or VST.

11. The method of claim 1, wherein the patient has a secondary immunodeficiency that comprises infection by HIV, a burn, drug abuse, chemotherapy, radiation therapy, diabetes millitus, malnutrition, or leukemia or other cancer of the immune system, viral hepatitis or other immune complex disease, or multiple myeloma.

12. The method of claim 1, wherein the NN includes a first NN model and a second NN model cascaded to the first NN model, and inputting the values for the one or more variables to the NN to produce the score includes:
inputting the values for the one or more variables to the first NN model to generate synthetic data that are in a larger amount than the values of the one or more variables; and
inputting the synthetic data to the second NN model to produce the score.

13. The method of claim 12, wherein the first NN model comprises a generative artificial intelligence (genAI) model, wherein the genAI model comprises a variational autoencoder (VAE) model, a generative adversarial network (GAN) model, or a Gaussian copula synthesizer (GC) model.

14. The method of claim 12, wherein the second NN model comprises a logistic regression (LR) model, a naïve Bayes (NB) model, and/or a support vector machine (SVM) model.

15. The method of claim 12, further comprising: determining similarity of the synthetic data to the original data, wherein the second NN model is trained with the synthetic data if the similarity of the synthetic data is over a similarity threshold in terms of the distribution to the original data.

16. The method of claim 15, wherein the similarity of the synthetic data is accessed by Total Variation Distance complement, Kolmogorov-Smirnov complement, or Spearman correlations.

17. The method of claim 1, further comprising: identifying at least one of the variables that contributes most to a predictive ability of the therapeutic approach.

18. The method of claim 12, further comprising:
inputting training values for the one or more variables to the first NN model to generate training synthetic data that are in a larger amount than the training values of the one or more variables; and
training the second NN with the training synthetic data.

19. The method of claim 1, wherein the one or more variables include continuous, binary and/or categorical variables.

20. The method of claim 19, wherein the values of the categorical variables are one-hot encoded prior to modeling.

21. The method of claim 19, wherein the values of the continuous variables are log normalized.

22. A method for treating an immunocompromised patient in need of an anti-cancer medication and/or in need of an anti-viral medication comprising:
(a) collecting from patient values for one or more variables, comprising selecting prior cancer remission or relapse, prior reaction after transplant, a degree of HLA match, type of viral infection, type of comorbidity or infection, viral load, prior receipt of one or more immunosuppressive medications, prior receipt of one or more anti-cancer medications, or prior receipt of one or more antiviral medications
(b) inputting the values for the one or more variables to a neural network (NN) performed on one or more computers to produce (i) a score indicative of likelihood of a therapeutic response, non-response, or anti-therapeutic response to an anti-viral drug and/or (ii) a score indicative of likelihood of a therapeutic response or non-response to Virus-Specific T-Cells (VSTs); and
(c1) administering an antiviral drug to a patient who has a threshold score indicative of a likelihood of a therapeutic response to anti-viral therapy, and/or
(c2) administering an anti-cancer drug to a patient who has a threshold score indicative of a likelihood of a therapeutic response to anti-cancer therapy; and/or
(c3) administering VSTs to a patient who has a threshold score indicative of a likelihood of a therapeutic response to VST therapy.

23. The method of claim 22, further comprising:
(a) collecting from the patient values for at least one variable selected from the group comprising:
prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor, or
prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir;
(b) inputting the values for the one or more variables to a neural network (NN) performed on one or more computers to produce (i) a score indicative of likelihood of response, non-response, or anti-therapeutic response to an anti-viral drug and/or (ii) a score indicative of likelihood of response, non-response, or antitherapeutic response to Virus-Specific T-Cells (VSTs); and
(c) administering an antiviral drug to a patient who has a threshold score indicative of a likelihood of a therapeutic response to anti-viral therapy; and/or
administering VSTs to a patient who has a threshold score indicative of a likelihood of a therapeutic response to VST therapy.

24. The method according to claim 22, wherein the group further comprises:
presence or absence of inborn error of immunity, malignant, non-malignant hematology condition, or other diagnosis, upon original diagnosis,
presence or absence of partial or complete cancer remission including no detectable cancer, reduction or growth of a tumor, higher or lower number of cancer cells compared to prior levels, and symptomatic improvement or regression compared to a prior level,
prior cancer relapse, transplant donor and recipient age and sex,
prior graft-vs-host reaction after transplant, or
myeloablative conditioning regiment (MA), reduced intensity conditioning regimen RIC), or no conditioning regiment (NMA),
transplant donor type including mismatched related donor, matched related donor, matched unrelated donor, umbilical cord cell transplant, or no donor.

25. The method according to claim 22, wherein the group further comprises:
a degree of HLA match ranging from 1 to 6 based on the number of major alleles shared, wherein said major alleles include HLA-A, HLA-B, HLA-C and HLA-DR, HLA-DQ and HLA-DP,
cellular depletion or ablation of TCRαβ, CD19, naive T cells (CD45RA+ T cells) and/or CD34+ T cells,
a level of CD8+ or CD8+ T cells or a ratio of CD4+ cells to CD8+ T cells or a higher or lower level compared to a prior level,
a type of viral infection comprising adenovirus (AdV), Epstien-Barr Virus (EBVCytomegalovirus (CMV), Herpes Simplex Virus (HSV), human herpes virus 8, Varicella-Zoster virus, or human papillomarvirus, or
a type of comorbidity or infection caused by an opportunistic bacterium, fungi, or parasite, a viral load at a time of infusion measured in IU/ml by PCR.

26. The method according to claim 22, wherein the variable group further comprises:
prior receipt of one or more immunosuppressive medication comprising systemic corticosteroids, Budesonide, Tacrolimus (FK), Cyclosporine (CsA), Mycophenolic acid (MMF), Sirolimus, Anti-thymocyte globulin (ATG), Alemtuzumab (Campath), or prior receipt of one or more antivirals including Ganciclovir, Valganciclovir, Foscarnet, Cidofovir, Brincidofovir, and Rituximab.

27. The method according to claim 22, wherein the variable group further comprises:
prior receipt of one or more immunosuppressive medication comprising systemic corticosteroids, Budesonide, Tacrolimus (FK), Cyclosporine (CsA), Mycophenolic acid (MMF), Sirolimus, Anti-thymocyte globulin (ATG), or Alemtuzumab (Campath),
prior receipt of one or more antivirals including Ganciclovir, Valganciclovir, Foscarnet, Cidofovir, Brincidofovir, and Rituximab,
prior receipt of one or more anti-cancer medications comprising azacitidine, doxorubicin, fludarabine, capecitabine, methotrexate, pembrolizumab, cyclophosphamide, clofarabine, fluorouracil, mercaptopurine, altretamine, bendamustine, busulfan, carboplatin, dacarbazine, daunorubicin, floxuridine, gemcitabine, trastuzumab, hydroxyurea, ifosfamine, melphaslan, nivolumab, paclitaxel, or other anticancer or checkpoint inhibitor, and
prior receipt of one or more antiviral medications comprising oseltamivir, acyclovir, entecavir, peramivir, valacyclovir, amantadine, famciclovir, ribavirin, adefovir, emtrictabine, foscarnet, ganciclovir, lamivudine, telbivudine, zanamivir, zanamivir, baloxavir marboxil, brivudine, cidofovir, laninamivir, sofosbuvir, or tenofovir.
